(19) United States
(12) Patent Application Publication
(10) Pub. No.: US 2015/0150982 A1
(43) Pub. Date: Jun. 4, 2015

(54) PHARMACEUTICAL FORMULATION FOR A THERAPEUTIC ANTIBODY

(71) Applicant: Boehringer Ingelheim International GmbH, Ingelheim am Rhein (DE)

(72) Inventors: Frank Michael, Ummendorf (DE); Stefan Bassarab, Biberach an der Riss (DE); Anna Maria Eibofner, Eberhardzell (DE); Silke Muehlau, Biberach an der Riss (DE); Markus Wolfram, Biberach an der Riss (DE)

(21) Appl. No.: 14/406,832

(22) PCT Filed: Jun. 12, 2013
(86) PCT No.: PCT/EP2013/062063

§ 371 Date: Dec. 10, 2014

(30) Foreign Application Priority Data
Jun. 12, 2012 (EP) 12171617.9

(51) Int. Cl. A61K 47/26; A61K 47/10; A61K 9/00; A61K 47/14; A61K 47/18; C07K 16/24; A61K 47/22; A61K 47/12

(52) U.S. Cl. CPC A61K 47/26; A61K 47/22; A61K 47/10; A61K 47/12; A61K 47/14; A61K 47/183; C07K 16/241; A61K 9/0019; C07K 2317/55

(57) ABSTRACT

The present invention relates to pharmaceutical formulations for a therapeutical antibody, preferably an IgG, said formulation comprising at least acetate/acidic acid, arginine, and trehalose. In addition, the present invention relates to pharmaceutical formulations for a therapeutical antibody, preferably an IgG, said formulation comprising at least histidine, mannitol and/or succinate and trehalose.

### FIELD OF THE INVENTION

The present invention relates to a pharmaceutical formulation for a therapeutic antibody

### BACKGROUND OF THE INVENTION

Therapeutic antibodies are large and complex molecules and, as such, subject to degradation processes, particularly in liquid state. While antibody production and purification is a well-controlled process, stable formulation and delivery is an issue. Physical and chemical instability of antibodies is really a complex function of solution conditions and temperature. Antibodies are for example susceptible to deamidation, isomerization, oxidation, proteolysis, aggregation and other covalent modifications. These phenomena are suspected to result in decreasing efficacy or even potential clinical side-effects or toxicity, since aggregates can reduce the efficacy and enhance the immunogenicity of the protein drug. Antibody aggregation is also a source of batch to batch variabilities in the antibody production chain and its control leads to regulatory and quality control burden which have extremely costly consequences. Further, aggregation of antibodies affects their stability in storage, including shelf-life and their useable administration time, once removed from optimum storage conditions.

An aqueous antibody formulation usually requires at least a buffer to maintain a given pH range, and a tonifier to ensure that the formulation has a similar osmolarity as physiological liquids. Typically, citrate and phosphate are used as buffers, while polyols like mannitol or salts like sodium chloride are used as tonifiers.

Two major indications of therapeutical antibodies are autoimmune diseases and cancers. While autoimmune diseases as such, chronic, many cancer types turn chronic or near-chronic due to targeted therapy. It is however desirable to provide therapeutic antibodies for the treatment of chronic diseases in a form that they can be administered by the patient himself (“home use”), because, in many cases, the drug will have to be administered life-long. The suitability of a formulation for self-administration will thus increase patient compliance and reduce costs, as the patient does not have to see medical personnel each time he need his drug injected.

In solutions, which are not stored at optimum conditions, such as at increased temperatures above the recommended range of 2-8° C., unwanted degradation occurs, which includes the formation of insoluble and/or soluble aggregates. Those insoluble and soluble aggregates are likely to be formed in the liquid state by association of the antibody molecule. In cases when a liquid formulation is stored for a long period of time, the bioactivity of the antibody molecules might get lost or reduced due to deamidation of aspargine residues. The cycle of freezing and thawing may also lead to the formation of degraded and aggregated antibody molecules.

As a result the solutions may exhibit lowered activity, increased toxicity, and/or increased immunogenicity. Indeed, polypeptide precipitation can lead to thrombosis, non-homogeneity of dosage form, and immune reactions. Thus, the safety and efficacy of any pharmaceutical formulation of a polypeptide is directly related to its stability.

However, the suitability for self-administration creates new challenges with respect to shelf life. The patient will have to store considerable amounts of drug at home, where storage conditions are often less suitable than in a medical practice. A formulation comprising a therapeutic antibody, which is suitable for self-administration will thus have to exceed existing formulations in terms of storage stability even under suboptimal conditions, e.g. break in the cool chain and the like. Thus, there is a need for stable, antibody formulations that provide dosing advantages and administrative advantages, particularly with respect to improved stability in storage, including shelf-life and their useable administration.

The above-mentioned problems are solved by the embodiments characterized in the claims and described further below.

### SUMMARY OF THE INVENTION

The invention relates to pharmaceutical composition, wherein the composition comprises an essentially citrate- and/or phosphate-free buffer solution designed for administration of a therapeutically active antibody or antibody fragment thereof, wherein the antibody is used for the treatment of autoimmune or malignant diseases, the buffer comprising and/or essentially consisting of: greater than 5 mg/ml antibody, and having a pH value of at least 5.5, preferably of 6.25+/−0.5.

The present invention is based on the surprising finding that pharmaceutical formulations having a pH value of 6.25+/−0.5 and being essentially free of citrate-phosphate as well as NaCl improve the long-term stability of an antibody or antibody fragment or antigen-binding fragment thereof by preserving its binding activity

Thus, the present invention relates to a buffer, wherein the solution comprising at least:

i) histidine and mannitol in pharmaceutically acceptable quantities; or
ii) succinate and trehalose in pharmaceutically acceptable quantities; or
iii) acetate or acidic acid, arginine, and trehalose in pharmaceutically acceptable quantities and at a pharmaceutically acceptable pH.

In a preferred embodiment of the present invention the therapeutic antibody is an anti-TNF alpha antibody, and the composition comprising:
i) the anti-TNF alpha antibody in a concentration of 50 mg/ml, histidine in a concentration of 25 mmol l⁻¹, and mannitol in a concentration of 240 mmol l⁻¹; or
ii) the anti-TNF alpha antibody in a concentration of 50 mg/ml, succinate in a concentration of 25 mmol l⁻¹, and trehalose in a concentration of 215 mmol l⁻¹; or
iii) the anti-TNF alpha antibody in a concentration of 50 mg/ml, acetate and/or acidic acid in a concentration of 15 mmol l⁻¹, arginine in a concentration of 10 mmol l⁻¹ and trehalose in a concentration of 240 mmol l⁻¹; wherein the compositions of i) and ii) having a pH value of 6.25+/−0.5 and the composition in iii) having a pH value of 6.5+/−0.5.

In a further preferred embodiment of the present invention, is the therapeutic antibody or said anti-TNFalpha antibody selected from the group consisting of adalimumab, infliximab, certolizumabpegol, golimumab, and antibodies being biosimilar or interchangeable with respect to adalimumab, infliximab, certolizumabpegol, or golimumab. Further preferred is the pharmaceutical composition adapted for subcutaneous administration.

As could be demonstrated in the examples, the composition of the present invention is advantageous over a reference pharmaceutical composition as described in WO2004/016286 and distributed by the company Abott. In particular, the pharmaceutical composition (F3, F4, and/or F8) is more stable as (the originator) F1. Thus, in a preferred embodiment, the present invention relates to a pharmaceutical composition which is stable at 40° C.+/−3° C. for at least 3 months, preferably for at least 6 months.

In addition, in one embodiment according to the present invention, the pharmaceutical composition of the present invention, has at least one feature selected from the group consisting of: increased shelf life, better temperature stability, and/or decreased formation of aggregates, compared to a formulation comprising citrate and phosphate as buffers, and mannitol as tonifier.

As shown in the Examples and demonstrated in the Figures the pharmaceutical composition i) and/or iii) exhibiting a Z-average (nm) of below 12+/−1 and a PDI of below 0.6+/−0.2 and/or wherein said composition is substantially free from particulates upon storage at about 5° C. for at least 6 months as determined by visual inspection.

In a further preferred embodiment of the present invention the pharmaceutical composition i) and/or ii) exhibiting a turbidity value in FormazinNephelometry Units (FNU) below 10 and/or composition ii) exhibiting a turbidity below 20 FNU.

Furthermore, in one embodiment of the present invention, the antibody or fragment thereof in the composition retains at least 90% of binding ability to a TNF alpha and/or CD16 polypeptide compared to a reference antibody preparation.

In a further preferred embodiment of the present invention, less than 5%+/−0.5% of said antibody or fragment thereof forms an aggregate upon storage at about 40° C. for at least 6 months as determined by HP SEC.

In a further aspect of the present invention, the pharmaceutical composition is designed for the subcutaneous administration and/or for use in the treatment of a disease selected from the group consisting of autoimmune disorders and malignant diseases.

Since it is wildly known that citrate and/or phosphate buffers causes pain upon subcutaneous injection, the present invention relates in one embodiment to a pharmaceutical composition of the present invention, wherein injection of the composition reduces pain associated with the injection designed to be administrated in a subject, preferably when compared to injection of an formulation that essentially consisting of a citrate-phosphate buffer, mannitol, NaCl, polysorbate 80 at a pH 5.2.

In another aspect, the present invention relates to a preconfectioned injection device comprising an aqueous buffer solution or a pharmaceutical composition of the present invention.

The present invention also provides in another aspect a kit of parts, comprising at least a container comprising a pharmaceutical composition of present invention, and an injection device.

### BRIEF DESCRIPTION OF THE DRAWINGS

FIG. 1: Turbidity analysis (Stability Study at 40° C.). The turbidity of the formulation samples was measured informazinenephelometric units (FNU) at 0, 4, 8, 12, and 24 weeks. The turbidity was determined by light scattering from undiluted samples at a volume of 120 μL in duplicates using a turbidity photometer (Co. Boehringer-IngelheimPharma GmbH & Co. KG in collaboration with Co. Microparts) at a wavelength of 633 nm. The turbidity of each formulation was calculated as the mean of the duplicates. Under the given experimental conditions (storage for 0-24 weeks at a temperature of 40° C.) turbidity values for formulations F3 and in particular F4 and F8 are lower as compared to the originator formulation F1.

FIG. 2: Monomer Analysis (Stability study at 40° C.). The stability of the antibody was monitored using high performance liquid chromatography-size exclusion chromatography (HPLC-SEC) to determine if this protein will fragment or aggregate under the conditions tested. A TSKgel G3000SWXL (300×7.8 mm) analytical HPLC column was used with a running buffer consisting of 100 mM phosphate+200 mM L-arginine, pH 6.8. The monomer content is reported as the percent of the total area of all species detected. The data demonstrate that the decrease in monomer content over 24 weeks at 40° C. is slower for F3, F4 and F8 formulations than that of F1.

FIG. 3: Aggregate Analysis (Stability study at 40° C.). The stability of the antibody was monitored using high performance liquid chromatography-size exclusion chromatography (HPLC-SEC) to determine if this protein will fragment or aggregate under the conditions tested. A TSKgel G3000SWXL (300×7.8 mm) analytical HPLC column was used with a running buffer consisting of 100 mMphosphate+200 mM L-arginine, pH 6.8. The aggregate content is reported as the percent of the total area of all species detected. The aggregate content increases at a temperature of 40° C. over the course of 24 weeks in all formulations^{o} F8 exhibits smallest aggregate fractions (lowest increase over time at 24 weeks at 40° C.) followed by F3, F4 and F1.

FIG. 4: Fragment Analysis (Stability study at 40° C.). The stability of the antibody was monitored using high performance liquid chromatography-size exclusion chromatography (HPLC-SEC) to determine if this protein will fragment or aggregate under the conditions tested. A TSKgel G3000SWXL (300×7.8 mm) analytical HPLC column was used with a running buffer consisting of 100 mMphosphate+200 mM L-arginine, pH 6.8. The fragment content is reported as the percent of the total area of all species detected. The fragment content increases at a temperature of 40° C. over the course of 24 weeks in all formulations. F3 exhibits the lowest fragment fractions at 24 weeks at 40° C. followed by F8, F4 and F1.

FIG. 5: Particle Size Analysis (Stability study at 40° C.). Mean particle sizes (Z-average values) in the formulations F1, F3, F4, and F8 were determined by dynamic light scattering using a ZetasizerNanoZS ZEN3600 (Malvern Instruments). Two undiluted samples at a volume of 75 μL in a single-use cuvette were irradiated with a helium-neon laser at a wavelength of 633 nm and a temperature of 20° C. Mean particle sizes of all formulations do not change over the course of 24 weeks when stored at a temperature of 40° C. Mean particle sizes of formulation F1 and F3 are greater as compared to those in formulations F4 and F8. The PDI increased at 24 weeks only in formulation F1.

FIG. 6: Activity Analysis (Stability study at 40° C.). Protein function was monitored at different storage conditions by diluting the samples to a concentration of 0.6 mg/mL in assay buffer (0.01 M HEPES, 0.15 M NaCl, 3 mM EDTA and 0.005% PS-20). Three different types of binding were monitored, Protein A binding 6b was measured to determine if the protein was correctly folded and functional, then both CD16 6a and TNFα 6c binding were measured for functional activity. This testing was carried out using a Biacore instrument (GE Healthcare). At temperatures 40° C., binding properties of formulation F3, F4, and F8 are higher as compared to F1 over the course of 24 weeks.

FIG. 7: Overview: Comparison of formulations (stability) at t=12 weeks at 2-8° C. In contrast to F1, formulations F3, F4, and F8 exhibit no visible particles and in comparison to F1 all formulations exhibit lower turbidity values.

FIG. 8: Overview: Comparison of formulations (stability) at t=24 weeks at 40° C. In comparison to F1 all formulations exhibit lower turbidity values and higher monomer values. In addition CD16 binding activity was higher in all formulations as compared to F1.

### DETAILED DESCRIPTION OF THE INVENTION

The present invention is based on the surprising finding that pharmaceutical formulations having a pH value of 6.25+/−0.5 and being essentially citrate- and/or phosphate buffered free, improve the long-term stability of glycoproteins such as antibodies or antibody fragment or antigen-binding fragment thereof by preserving its binding activity. The formulations of the invention provide a number of surprising characteristics given the high stability at ambient temperatures of the therapeutic antibody. In particular, all formulations of the present invention exhibit superior turbidity values, high content of monomeric antibodies, less aggregates and fragments, even when the formulation has been even stored for up to six month at 40° C.+3° C.

In addition, the antibody formulated in the pharmaceutical compositions of the present invention exhibit a far better binding activity and/or stability after storage of 24 weeks at 40° C. as measured by Biacore than a comparison formulation of WO2004/016286. Furthermore, injection of the compositions of the present invention may exhibit less irritation of the skin compared to the to a citrate-phosphate buffer comprising sodium chloride. Thus, it is tempting to speculate that these effects are due to the specific pH value of 6.25 or 6.5 of the pharmaceutical described herein.

Put in other words, the present invention relates to an essentially citrate and/or phosphate-free aqueous buffer solution for administration of a therapeutically active antibody or antibody fragment thereof, wherein the antibody is used for the treatment of autoimmune or malignant diseases, the buffer comprising: greater than 5 mg/ml antibody, and having a pH value of at least 5.5, preferably of 6.25+/−0.5.

As used herein the term “essentially” denotes a composition or formulation or buffer or solution wherein no citrate and/or phosphate molecules are actively, i.e. intended to be added. Trace amounts of citrate and/or phosphate may be present in a concentration below 3 mM, 2 mM, 1 mM, preferably below 0.5 mM, more preferably below 0.05 mM.

According to the present invention, a pharmaceutical formulation for a therapeutical antibody or antibody fragment is provided, said formulation comprising at least: i) histidine, and mannitol in pharmaceutically acceptable quantities; or ii) succinate, and trehalose in pharmaceutically acceptable quantities; or iii) acetate, or acidic acid arginine, and trehalose in pharmaceutically acceptable quantities and at a pharmaceutically acceptable pH.

The formulation according to the present invention has improved properties compared to other formulations existing in the art, as will be described below. The antibody is preferably a monoclonal antibody and more preferably an IgG format antibody.

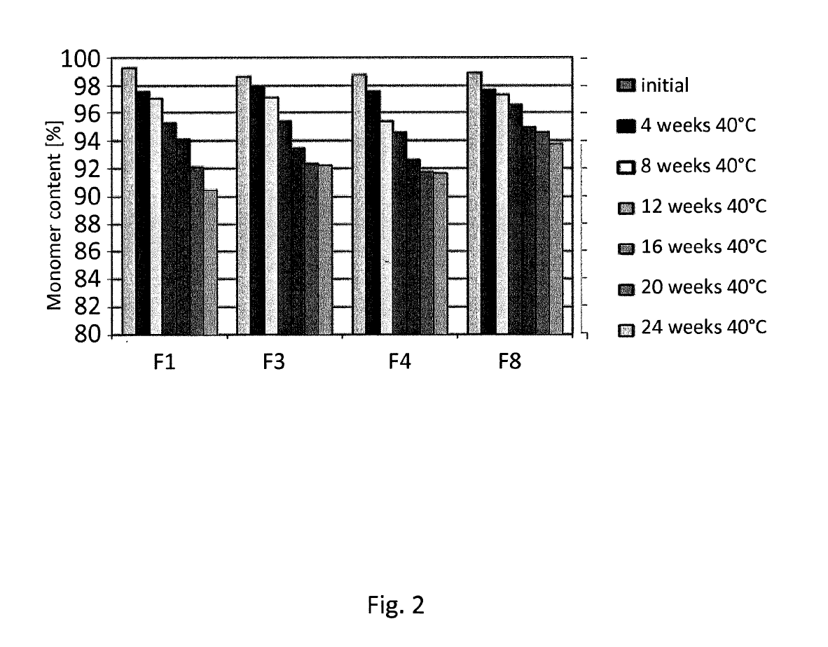

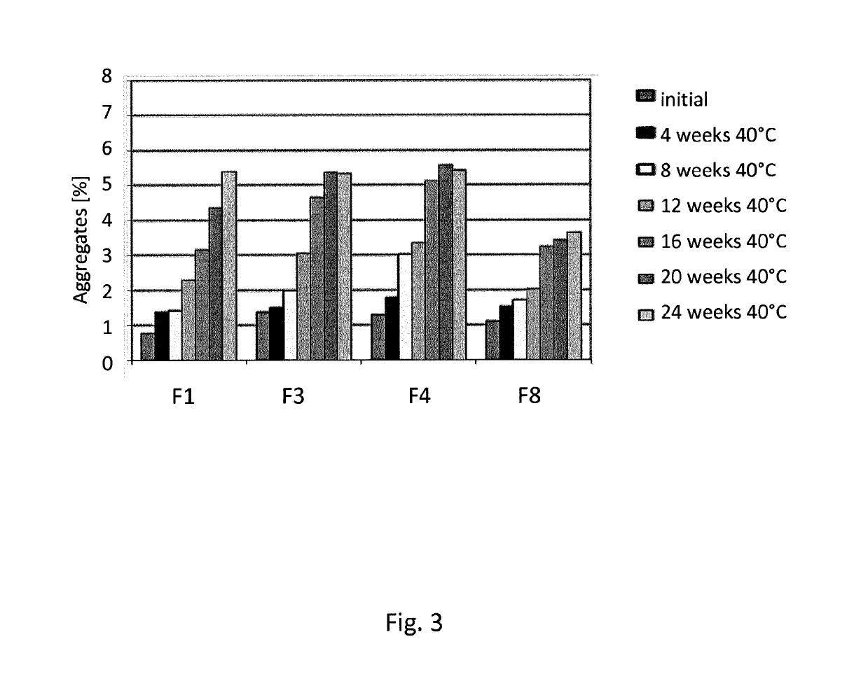

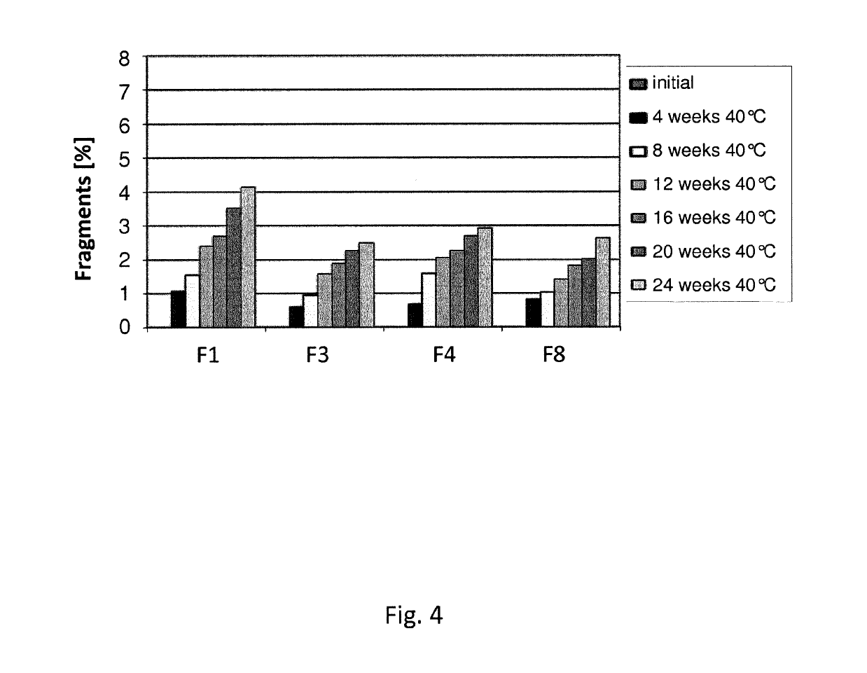

The compositions of this invention minimize the formation of antibody aggregates and particulates and insure that the antibody maintains its bioactivity over time. In particular the inventors of the present invention made the surprising finding as demonstrated in the Examples 1 and 2 as well as in the FIGS. 2 and 4 that the content of monomers of the formulated antibody is more stable and the formations of fragments are far less pronounced in the compositions of the present invention at 40° C. when stored for 3 weeks or three month or even six month compared to a formulation containing citrate and phosphate buffer. As shown in the Example 1 and 2 the F1 formulation exhibiting a pH value of 5.2 discloses a high initial value of turbidity and slightly increase compared to the formulations F3, F4 and/or F8, which likely reflects protein aggregation. Removing this aggregation requires additional steps such as a newly addition of Polysorbate 80 and subsequently sterile filtration including loss of the initial antibody concentration.

As a result, in one aspect of the present invention provides a pharmaceutical composition comprising i) a therapeutic antibody or antibody fragment thereof in a concentration of from 5 to 200 mg/ml, preferably from 10 to 150 mg/ml, histidine in a concentration of from 10 to 200 mmol l⁻¹, preferably from 10 to 50 mmol l⁻¹, mannitol in a concentration of from 50 to 1000 mmol l⁻¹ preferably from 100 to 400 mmol l⁻¹, and optionally a surfactant, at a pH value in the range of 5.5 to 8, of 6 to 7, preferably of 6.1 to 6.7, or ii) a therapeutic antibody or antibody fragment thereof in a concentration of from 5 to 200 mg/ml, preferably from 10 to 150 mg/ml, succinate in a concentration of from 10 to 200 mmol l⁻^{₁}, preferably from 10 to 50 mmol l⁻¹, trehalose in a concentration of from 50 to 1000 mmol l⁻¹, preferably from 100 to 400 mmol l⁻¹, and optionally a surfactant, at a pH value in the range of 5.5 to 8, of 6 to 7, preferably of 6.1 to 6.7; or iii) a therapeutic antibody or antibody fragment thereof in a concentration of from 5 to 200 mg/ml, preferably from 10 to 150 mg/ml, acetate and/or acidic acid in a concentration of from 10 to 200 mMol l⁻¹, preferably from 10 to 50 mMol l⁻¹, arginine in a concentration of from 5 to 100 mMol l⁻¹, and preferably from 5 to 40 mMol l⁻¹; trehalose in a concentration of from 50 to 1000 mMol l⁻¹, and preferably from 100 to 400 mMol l⁻¹, and optionally a surfactant, at a pH value in the range of 5.5 to 8, preferably of 6 to 7.

The term “therapeutical antibody” as used herein comprises human, humanized, chimeric and murine antibodies. It further comprises native antibodies isolated from man, mammals, vertrebrates or chordates as well as mutagenized or genetically engineered antibodies. As regards the term “IgG format”, it should be noted that said term covers, among others, the different IgG subclasses (e.g.; IgG1, 2, 3, and 4). IgG antibodies are molecules of about 150 kDa composed of four peptide chains. It contains two identical class gamma heavy chains of about 50 kDa and two identical light chains of about 25 kDa, thus a tetrameric quaternary structure. The two heavy chains are linked to each other and to one light chain each by disulfide bonds. The resulting tetramer has two identical halves, which together form a fork, or a Y-like shape. Each end of the fork contains an identical antigen binding site. The Fc regions of IgGs bear a highly conserved N-glycosylation site. The N-glycans attached to this site are predominantly core-fucosylatedbiantennary structures of the complex type. In addition, small amounts of these N-glycans also bear bisecting GlcNAc and alpha-2,6-linked sialic acid residues. In a preferred embodiment of the present invention, acetate and arginine act as buffers, and trehalose acts as a tonifier. The term “therapeutical antibody” additionally encompasses non-IgG format antibodies, such as IgM antibodies, single domain antibodies, and the like. The term “(therapeutical) antibody fragments” includes Fab fragments, Fv fragments, single chain Fv (scFv) fragments, and the like, optionally in a modified form, e.g. to increase serum half-life thereof.

Acetic acid is an organic acid abbreviated as CH3COOH. Its deprotonized form, acetate, is abbreviated as CH3COO⁻. Acetic acid is a weak monoprotic acid which has, in aqueous solution, a pKa value of 4.75.

Arginine, abbreviated as Arg, is a proteinogenic alpha-amino acid having basic chemical properties. Its side-chain consists of a 3-carbon aliphatic straight chain, the distal end of which is capped by a complex guanidinium group, which imparts basic chemical properties to arginine due to a pKa value of 12.48. Arginine and acetate, or acetic acid, form a non-standard buffer in the formulation according to the present invention. Such buffer system is needed to provide optimal antibody stability under conditions to which the formulation will be exposed. In some formulations, freezing may lead to a pH shift, while in other cases; molarity caused by buffers may lead to antibody aggregation. Further, the pH of the formulation may change due to degradation of its ingredients. pH shifts beyond an accepted range are however detrimental, as they can lead to inactivation or even denaturation of the antibody, or render the formulation physiologically unacceptable.

Trehalose is an alpha-linked disaccharide formed by an alpha,alpha-1,1-glucoside bond between two alpha-glucose units (alpha-D-glucopyranosyl-(1→1)-alpha-D-glucopyranoside). It finds use in biotechnology to preserve proteins and nucleic acids, for example on biochips. In the present formulation, trehalose acts as a tonifier, i.e., a tonicifying agent, which serves to adjust the tonicity, or osmolarity, of the aqueous formulation. In one embodiment, the tonifier used in the formulation or methods of the invention is mannitol.

Succinate (pKa 5.63) is a preferred buffer for subcutaneous injection. Citrate and phosphate buffers are much less preferred because it causes a painful reaction when injected subcutaneously.

Histidine (pK 5.97) is a preferred buffer for subcutaneous, intramuscular and peritoneal injection. The advantage of histidine buffer is that 1 mmole of the histidine buffer only contributes 1 mOsm, whereas 1 mmole of the sodium succinate buffer contributes 3 mOsm. Because histidine buffer contributes less to the osmolarity, it allows more stabilizing excipients to be added to the formulation.

In a preferred embodiment of the present invention, the formulation is an aqueous solution. This is the favored way the formulation is put on the market, because such type of formulation is ready to use and requires no preparational steps by the applicant.

In another preferred embodiment of the present invention, the formulation is lyophilized. Such type of formulation may have an even longer shelf life. In this embodiment, trehalose does not only act as a tonifier, but also as a cryoprotectant.

In yet another preferred embodiment of the present invention, the formulation further comprises a surfactant. Said surfactant is, preferably, a polysorbate, e.g., which is an emulsifier derived from PEG-ylatedsorbitan (a derivative of sorbitol) esterified with fatty acids. This class of agents comprises, among others, polysorbates 20, 21, 40, 60, 61, 65, 80, 81, 85, and 120. More preferably, polysorbate 80 (polyoxyethylene(20)-sorbitan-monooleate) or polysorbate 20 (polyoxyethylene(20)-sorbitan-monolaurate) is used.

In the following table, typical concentration ranges of the formulation according to the invention are given:

TABLE 1
a: Typical composition of a formulation according to the invention

| Compound | Range | | preferred composition 1 | |
|---|---|---|---|---|
| acetate, or acidic acid | 10-200 | mMol l−1 | 15 | mMol l−1 |
| arginine | 5-100 | mMol l−1 | 10 | mMol l−1 |
| trehalose | 50-1000 | mMol l−1 | 240 | mMol l−1 |
| polysorbate 80 | 0.001%-10.0% | m/v | 0.1% m/v (=0.76 mmol−1) | |
| pH | 6-7 | | 6.5 | |
| therapeutic antibody | 5-200 | mg/ml | 50 | mg/ml |

b: Typical composition of a formulation according to the invention

| Compound | Range | | Preferred composition 2 | |
|---|---|---|---|---|
| succinate | 10-200 | mmol l−1 | 25 | mmol l−1 |
| trehalose | 50-1000 | mmol l−1 | 215 | mmol l−1 |
| polysorbate 80 | 0.001%-10.0% | m/v | 0.1% m/v (=0.76 mmol−1) | |
| pH | 6-7 | | 6.25 | |
| Therapeutic antibody | 5-200 | mg/ml | 50 | mg/ml |

c: Typical composition of a formulation according to the invention

| | | |
|---|---|---|
| Compound | Range | Preferred composition 3 |

| | | | | |
|---|---|---|---|---|
| histidine | 10-200 | mmol l−1 | 25 | mmol l−1 |
| mannitol | 50-1000 | mmol l−1 | 240 | mmol l−1 |

| | | | |
|---|---|---|---|
| polysorbate 80 | 0.001%-10.0% | m/v | 0.1% m/v (=0.76 mmol−1) |

| | | |
|---|---|---|
| pH | 6-7 | 6.25 |

| | | | | |
|---|---|---|---|---|
| therapeutic antibody | 5-200 | mg/ml | 50 | mg/ml |

In another preferred embodiment, the pharmaceutical formulation according to the invention comprises between ≧10 mg/ml and ≦200 mg/ml therapeutic antibody. More preferably, the pharmaceutical formulation according to the invention comprises between ≧20 mg/ml and ≦100 mg/ml therapeutic antibody. Even more preferably, the pharmaceutical formulation according to the invention comprises between ≧30 mg/ml and ≦70 mg/ml therapeutic antibody.

Thus, in one embodiment the present invention relates to a pharmaceutical composition, wherein the therapeutic antibody is an anti-TNF alpha antibody, and the composition comprising: i) the anti-TNF alpha antibody in a concentration of 50 mg/ml, histidine in a concentration of 25 mmol l⁻¹, and mannitol in a concentration of 240 mmol l⁻¹; or ii) the anti-TNF alpha antibody in a concentration of 50 mg/ml, succinate in a concentration of 25 mmol l⁻¹, and trehalose in a concentration of 215 mmol l⁻¹; o iii) the anti-TNF alpha antibody in a concentration of 50 mg/ml, acetate and/or acidic acid in a concentration of 15 mmol l⁻¹, arginine in a concentration of 10 mMol⁻¹, and trehalose in a concentration of 240 mmol l⁻¹; wherein the compositions of i) and ii) having a pH value of 6.25+/−0.5 and the composition in iii) having a pH value of 6.5+/−0.5.

According to another preferred embodiment, the pharmaceutical formulation as outlined above comprises a therapeutical antibody used for the treatment of autoimmune diseases such as antibodies binding to the targets CD11a (e.g. efalizumab), IL1b (e.g. canakinumab), IgE (e.g. omalizuzmab), a4-integrin (e.g. natalizumab), IL12/23 (e.g. ustekinumab), IL6R (e.g. tocilizumab), or tumor necrosis factor alpha (TNFalpha; e.g. adalimumab, infliximab, golimumab, certolizumabpegol), or used for the treatment of malignant diseases, such as antibodies binding to the targets CD20, Her2, EGFR, CD33, CD52, CTLA-4, or CD30.

Specifically preferred is a pharmaceutical formulation as described above including an antibody binding to an epitope of TNFalpha (also known as TNF, cachexin or cachectin). Tumor necrosis factor promotes inflammatory responses, which, in turn, are causing many of the clinical problems associated with autoimmune disorders such as rheumatoid arthritis, ankylosing spondylitis, inflammatory bowel disease, psoriasis, hidradenitissuppurativa and refractory asthma.

The mere binding of at least one epitope of Tumor Necrosis Factor alpha by means of an antibody in the meaning of the present invention inhibits the receptor binding reaction and thus opens a mechanism to treat the above mentioned disorders.

In another preferred embodiment, the heavy chain variable region and/or the light chain variable region of the therapeutically antibody have at least 70% sequence identity with the corresponding sequences of adalimumab and/or infliximab and/or golimumab and/or certolizumabpegol. Preferably, the sequence identity of the heavy chain variable region and/or the light chain variable region with the respective corresponding sequences is >70, 71, 72, 73, 74, 75, 76, 77, 78, 79, 80, 81, 82, 83, 84, 85, 863, 87, 88, 89, 90, 91, 92, 93, 94, 95, 96, 97, 98, 99, or 100%. Protein sequences of antibodies mentioned above are publicly available, e.g. referenced in WO2004/016286 (adalimumab/D2E7).

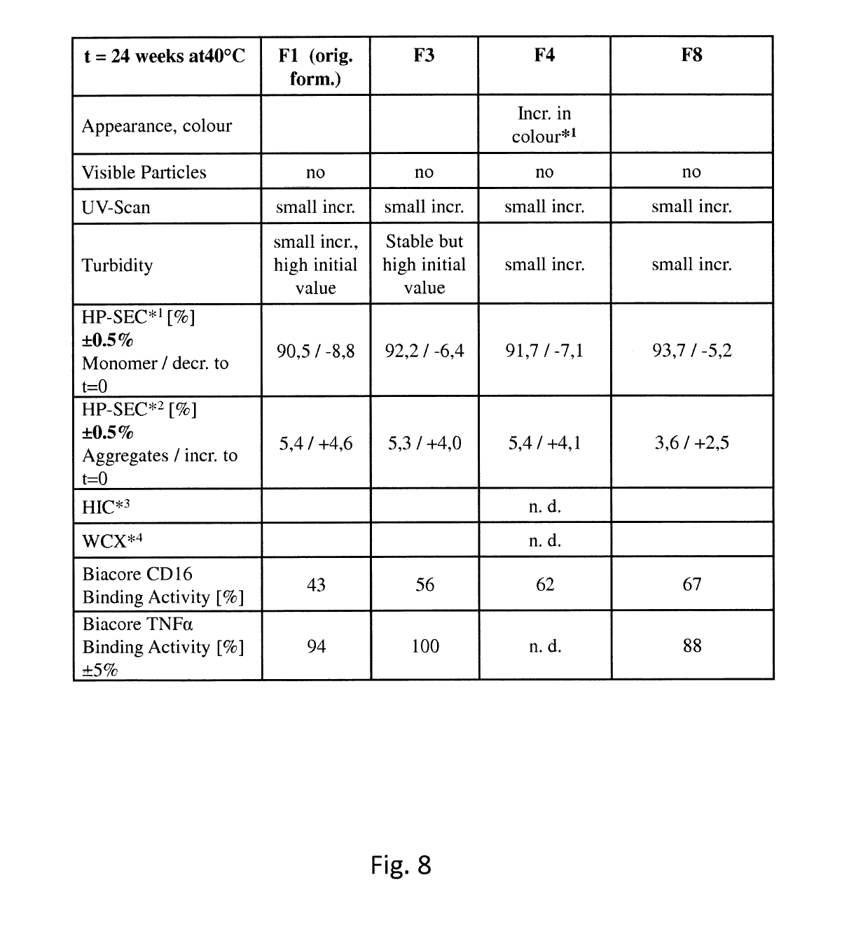

Several alternative buffer systems for the formulation of therapeutic useful antibodies such as TNF alpha exists, having also comparable features in terms of bioavailability and/or patient compliance and render them suitable as biosimilars. However, most of them suffer from the same disadvantages as the originator buffer as outlined above, due to the use of equivalent ingredients such as citrate and/or phosphate-buffered systems or using the same pH range. During the course of experiments performed in accordance with the present invention, the inventors of the present invention surprisingly discovered that by using an amino acid-buffered composition such as histidine and/or arginine provides pharmaceutical acceptable compositions suitable for the formulation of TNF alpha antibodies which could be equivalently used in terms of safety and efficiency as required by the FDA and the European Pharmacopeia as demonstrated in FIG. 8. In addition, also a succinate-based composition showed equivalent results when compared with the originator formulation during experiments performed at 2-8° C.

As used herein, “biosimilar” (of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biological product. In one embodiment, the biosimilar biological product and reference product utilize the same mechanism or mechanisms of action for the condition or conditions of use prescribed, recommended, or suggested in the proposed labeling, but only to the extent the mechanism or mechanisms of action are known for the reference product. In one embodiment, the condition or conditions of use prescribed, recommended, or suggested in the labeling proposed for the biological product have been previously approved for the reference product. In one embodiment, the route of administration, the dosage form, and/or the strength of the biological product are the same as those of the reference product. In one embodiment, the facility in which the biological product is manufactured, processed, packed, or held meets standards designed to assure that the biological product continues to be safe, pure, and potent. The reference product may be approved in at least one of the U.S., Europe, or Japan.

Thus, in one aspect, the compositions as named in accordance with the present invention F3, F4 and/or F8 exhibit more or less identical values at 2-8° C. However, surprisingly during the course of experiments performed in accordance with the present invention, the inventors discovered that the composition exhibit superior effects compared to the F1 composition.

Thus, in another preferred embodiment said formulation has at least one feature selected from the group consisting of increased shelf life, better temperature stability, and/or improved patient compliance compared to a formulation comprising citrate and phosphate as buffers, and mannitol and sodium chloride as tonifiers. See experimental section for further details.

As could be demonstrated in the Examples 1 and/or 2, the compositions of the present invention surprisingly exhibit an improved stability at high temperatures i.e. 40° C. for a long duration such as three months or even six months. It prudent to expect that the composition of the present invention, i.e. F3, F4 and/or F8 are even stable for up to one year at least at 2-8° C. and/or at about 40° C. Thus, it is tempting to speculate that due to the increased pH, stability of the formulations/buffer of the present invention are improved. Hence, in a preferred embodiment, the pharmaceutical composition is stable at 40° C.+/−3° C. for at least 3 months, preferably for at least 6 months. In accordance with the present invention the term “about” as used herein denotes the rage of the temperature given and a given temperature value can vary between 1, 2 and/or 3° C. In one embodiment according to the present invention, the pharmaceutical composition F3, F4 and/or F8 is more stable as the F1 formulation at 40° C.+/−3° C. for at least 3 months, preferably for at least 6 months.

It is well known that a citrate-buffered formulation of drugs as currently available on the marked increases pain upon subcutaneous injection see for example Kappelgaard A.-M. Horm Res. 2004; 62 Suppl 3:98-103. In a double-blind, randomized, placebo-controlled study the pain after subcutaneous injection of erythropoietin was mainly caused by the citrate component of the buffered solution (Frenken et at 1993). In addition, the impact of buffer concentration and pH has also been examined for a formulation of insulin-like growth factor I, in which a phosphate buffer was used. In that study high concentrations of buffer turned out to be the main responsible factor as concerns injection pain (Fransson&Espander—Jansson 1996). Since TNF-alpha treatment is perceptive to patients suffering from e.g. autoimmune diseases, these patient are even more sensitive to pain. Moreover, these patients need regularly treatments with duration of years. In addition, freezing phosphate buffers can lead to pH instability and can lead to increase aggregates. As a result, the use of phosphate-buffered formulation of drugs as currently available is also not preferred.

Thus, the present invention relates in one aspect to the provision of a formulation, buffer system composition, and/or solution, which will avoid local skin irritations and/or pain upon injection resulting in a dramatically increase of their quality of life and furthermore would help improving patient compliance. As can be seen from the results of the Examples described below, according to present invention, a stable formulation can be obtained in which not only aggregation, fragmentation and/or dimerization is reduced compared to the F1 formulation, which is essentially the citrate-phosphate-buffered formulation used in WO2004/016286, but even more surprisingly long-term storage at ambient temperature could be drastically increased.

In accordance with the above, in one embodiment the pharmaceutical composition of the present invention having at least one feature selected from the group consisting of:
(a) decreased amount of aggregates as measured by High Pressure Size Exclusion Chromatography (HP-SEC),
(b) higher amount of monomers after storage at about 40° C. as measured by HP-SEC
(c) significant less fragments as measured by HP-SEC,
(d) smaller hydrodynamic diameter in Z-Average (nm) at 24 weeks and/or less increase in Polydispersity Index (PDI) at storage at 40° C.
(e) increased binding activity as measured by Biacore after storage at about 40° C.,
(f) lower turbidity value in FormazinNephelometry Units (FNU), and/or
(g) less skin irradiation and/or reduced pain upon injection into a subject, compared to a reference composition.

In accordance with the present invention the terms “decreased”, “higher”, “less”, “smaller”, “increased”, “lower” or “less” the like, e.g., which denote quantitative differences between two states, refer to at least statistically significant differences between the two states.

In certain embodiments, the subject is human, or a non-human mammal. In certain embodiments, the reference composition or otherwise identical formulation is the commercially available adalimumab formulation of WO2004/016286 containing adalimumab, sodium chloride, monobasic sodium phosphate dihydrate, dibasic sodium phosphate dihydrate, sodium citrate, citric acid monohydrate, mannitol, polysorbate 80, and water for Injection.

In a preferred embodiment, the reference composition is a pharmaceutical composition comprising a TNF alpha antibody in citrate and phosphate as buffer, NaCl, mannitol as tonifier and at a pH of 5.2.

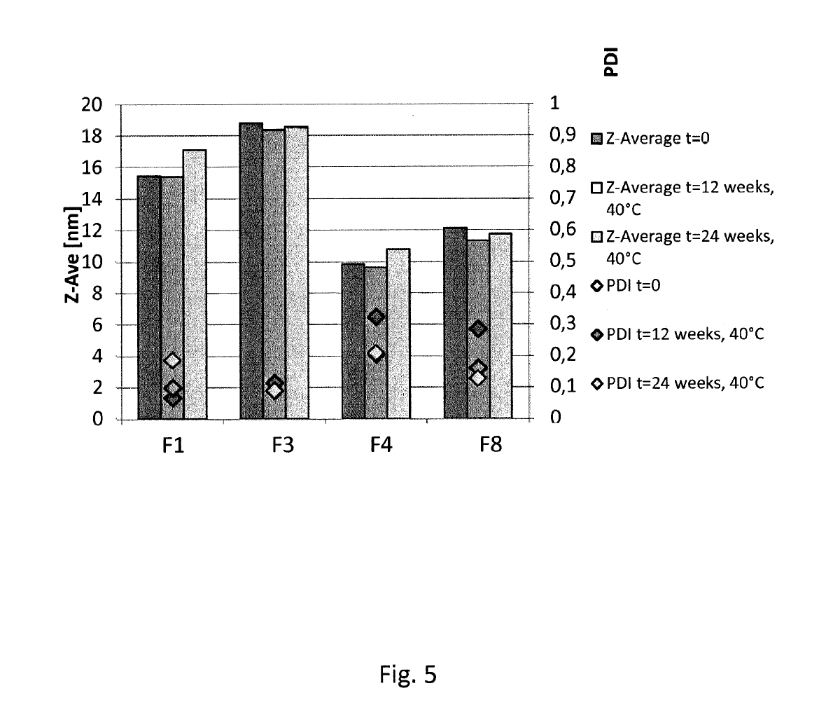

In one embodiment of the present invention, the pharmaceutical composition exhibit amount of mean particle size wherein composition i) and/or iii) exhibiting a Z-average (nm) of below 12+/−1 and a PDI of below 0.6+/−0.2 and/or wherein said composition is substantially free from particulates upon storage at about 5° C. for at least 6 months as determined by visual inspection. As outlined in the Examples 1 and/or 2 as well as in FIG. 5 the buffer compositions F3 and F8 surprisingly demonstrate that no change in hydrodynamic diameter at 24 weeks at 40° C. occur, and merely a very small increase could be detected for formulation F4. In contrast to that, the PDI of F1 increased over time, in particular at 24 weeks at 40° C. Thus, indicating the buffers exhibit improved stability compared to F1.

Visual inspection is well known to the skilled artisan and can be performed for example by measuring UV-scan in mg/ml or other known means and methods.

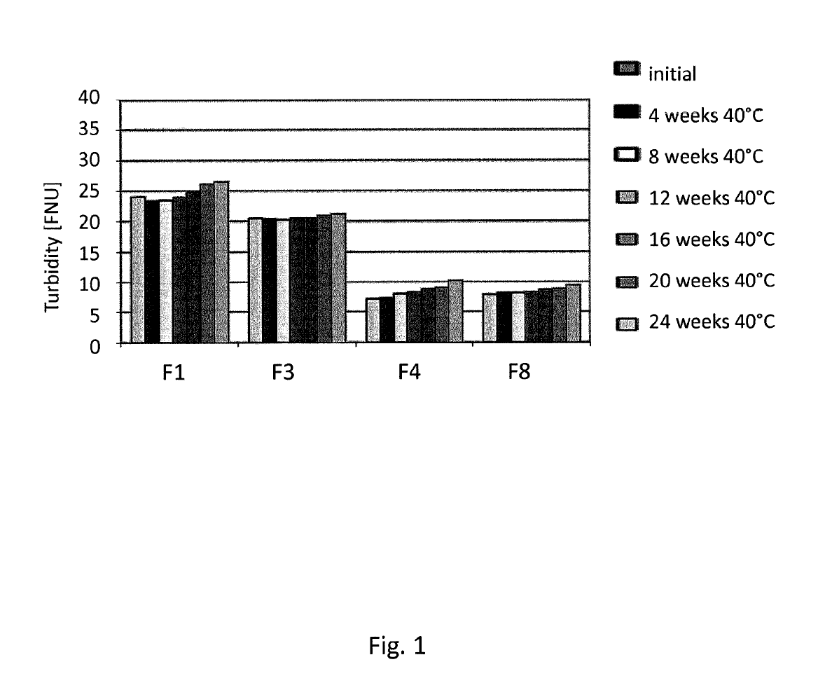

Furthermore, all compositions F3, F4 and F8 exhibit lower turbidity values than F1 at high temperatures, see Example 1 and/or 2 as well as FIG. 1. Hence, in a further embodiment of the present invention, the pharmaceutical composition i) and/or ii) exhibiting a turbidity value in FormazinNephelometry Units (FNU) below 10 and/or composition ii) exhibiting a turbidity below 20 FNU.

Of upmost importance for a suitable pharmaceutically active composition is the amount of monomeric antibodies formulated in the solution. Since aggregates are responsible for causing several as well as severe side effects, the content of monomers displays the actual pharmaceutically active amount of the drug, i.e. the antibody or antibody fragment thereof. In view to the above, a composition which is suitable for self-administration is always at the risk of improper storage condition such at high temperatures. As evident from the examples as well as FIG. 2 the monomer content of the compositions of the present invention is not only at about 99%+/−1% at 2-8° C. at 24 weeks but also above 91 to 92% even after long term storage at higher temperatures.

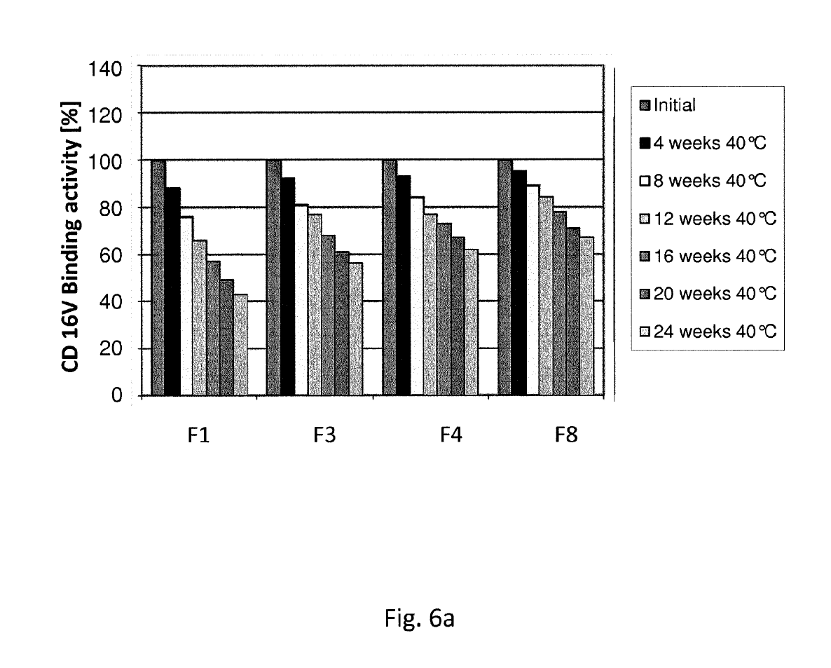

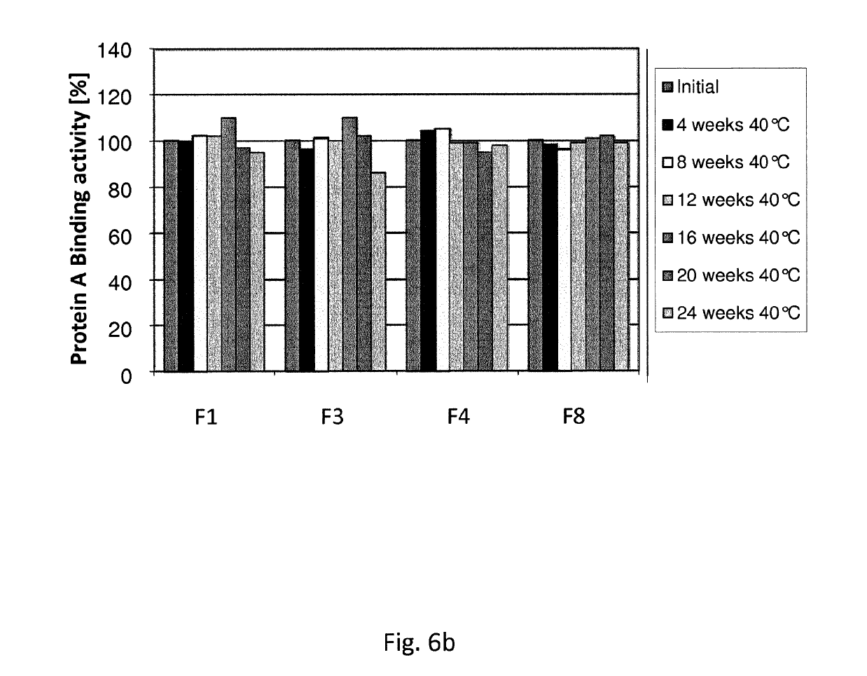

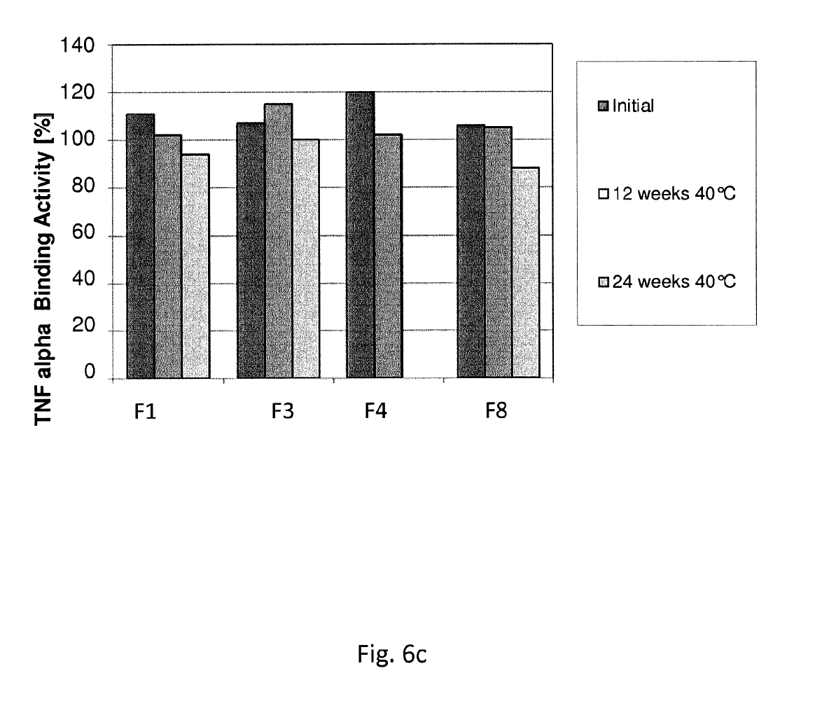

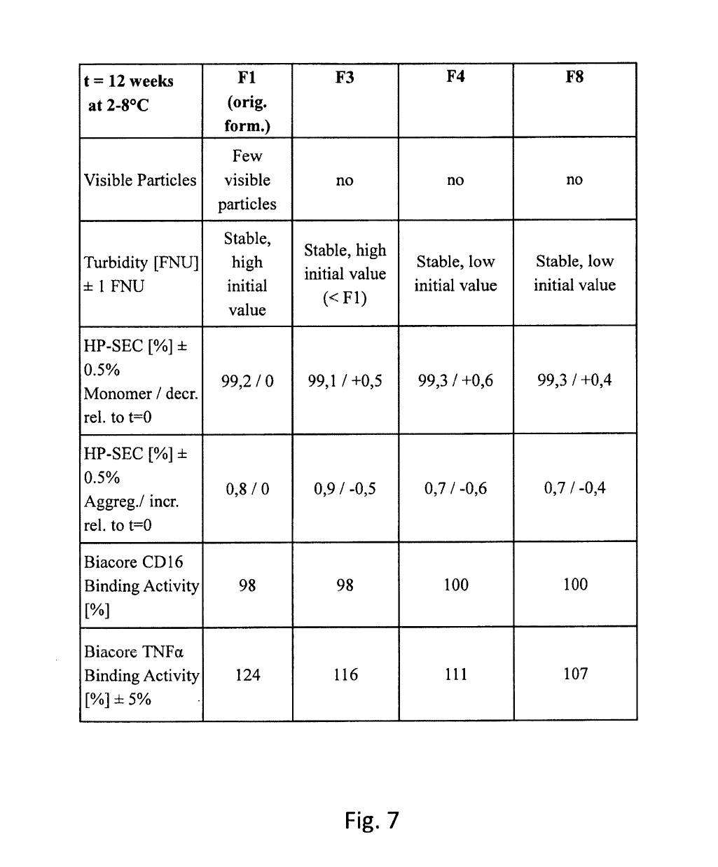

Thus, in a preferred embodiment of the present invention, said antibody or fragment thereof retains at least 90% of binding ability to a transforming growth factor alpha and/or CD16 polypeptide compared to a reference antibody preparation. This could be demonstrated in accordance with the present invention in Example 2 as well as FIGS. 6 to 8 relating to the binding activity of a TNF alpha antibody stored and/or formulated in the any one of the F3, F4 and/or F8 formulation.

In a further embodiment according the present invention, the pharmaceutical composition comprises less than 5%+/−0.5% of said antibody or fragment thereof forms an aggregate upon storage at about 40° C. for at least 6 months as determined by HP SEC.

Naturally the present invention extents to a pharmaceutical composition wherein less than 3%+/−0.5% of said antibody or fragment thereof is fragmented upon storage at about 40° C. for at least 6 months as determined by HP SEC.

In line with the above, in one embodiment of the present invention the pharmaceutical composition contains, essentially consists of or comprises less than 91.7%+/−0.5% of said antibody or fragment thereof is monomeric upon storage at about 40° C. for at least 6 months as determined by HP SEC.

A “stable” formulation, composition or solution in accordance with the present invention is one in which all the protein, i.e. antibody and/or antibody fragment thereof therein essentially retain their physical stability and/or chemical stability and/or biological activity upon storage at the intended storage temperature, e.g. 2-8° C. and/or 40° C. It is desired that the formulation essentially retains its physical and chemical stability, as well as its biological activity upon storage. The storage period is generally selected based on the intended shelf-life of the formulation.

A “more stable” pharmaceutical composition, formulation, buffer or aqueous solution in accordance with the present invention is one wherein the protein, i.e. antibody and/or fragment thereof denotes quantitative differences between two states, referring to at least statistically significant differences between the two states, wherein states in this context means the physical stability and/or chemical stability and/or biological activity of the protein, i.e. the antibody or antibody fragment thereof.

Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asndeamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation changes, etc. A “deamidated” monoclonal antibody herein is one in which one or more asparagine residue thereof has been modified, e.g. to an aspartic acid or an iso-aspartic acid by a post-translational modification.

In another preferred embodiment of the present invention, said formulation is adapted for subcutaneous administration. In subcutaneous administration, or injection, of a drug (abbreviated as SC or SQ) the drug delivers a bolus into the subcutis the layer of skin directly below the dermis and epidermis, collectively referred to as the cutis. Subcutaneous injections are highly effective, and well established, in administering medications such as insulin, as they can be performed by non-medically skilled persons provided they have received respective training because of reduced risk of infection and ease of administration. Subcutaneous administration is thus suitable for ambulant administration, administration in areas of poor infrastructure, e.g., where non-medically skilled persons are responsible for the drug administration, or home use.

The latter is particularly important in therapeutic regimens which require repeated treatment, as is the case in many chronic diseases, like autoimmune diseases (e.g., rheumatoid arthritis, ankylosing spondylitis, inflammatory bowel disease, psoriasis, hidradenitissuppurativa and refractory asthma) or in many cancer types which, due to targeted therapy, turn chronic or near-chronic.

However, for the above reasons formulations which are adapted for subcutaneous administration have a higher risk to be exposed to suboptimal storage conditions by ordinary persons, e.g., the cool chain is interrupted, or the formulations are exposed to light or sudden temperature changes. Furthermore, SC formulations require a relatively high concentration of the therapeutic agent, because the volume administered with one injection is rather limited (1.5 to a maximum of 2.0 mL). In addition, in order to reduce needle pain during injection, the needle needs to be thin, requiring a low viscosity of the injected solution. And finally, SC injection may result in pain at the injection site, even after the needle has been removed. This is probably influenced by components of the protein solution, such as the sort of buffer molecules and the osmolarity, and may have a significant influence on patient compliance of the respective therapy. The formulation according to the present invention, with its increased shelf life, better temperature stability, improved viscosity, and improved patient compliance is thus particularly suitable for subcutaneous administration.

In line with the above, the present invention relates in one embodiment to pharmaceutical composition, wherein injection of the composition reduces pain associated with the injection designed to be administrated in a subject, preferably when compared to injection of a formulation that essentially consisting of a citrate-phosphate buffer, mannitol, NaCl, polysorbate 80 at a pH 5.2.

Pain may be evaluated using any type of pain assessment known in the art, including, for example, visual analog scales, qualitative assessments of pain, or needle pain assessments. For example, subject-perceived injection site pain may be assessed using the Pain Visual Analog Scale (VAS). A VAS is a measurement instrument that measures pain as it ranges across a continuum of values, e.g., from none to an extreme amount of pain. Operationally a VAS is a horizontal line, about 100 mm in length, anchored by numerical and/or word descriptors, e.g., 0 or 10, or “no pain” or “excruciating pain,” optionally with additional word or numeric descriptors between the extremes, e.g., mild, moderate, and severe; or 1 through 9) (see, e.g., Lee J S, et al. (2000) AcadEmerg Med 7:550, or Singer and Thods (1998) Academic Emergency Medicine 5:1007). Pain may be assessed at a single time or at various times following administration of a formulation of the invention such as, for example, immediately after injection, at about 1, 2, 3, 4, 5, 6, 7, 8, 9, 10, 15, 20, 25, 30, 35, 40, or 45 minutes after injection.

In a certain embodiment of the invention, injection of the formulation into a subject results in a Pain Visual Analog Scale score of less than 0.6, 0.7, 0.8, 0.9, 1.0, 2.0, 3.0, 4.0, or 5.0 on a scale of 0 (no pain) to 10 (excruciating pain).

Other tools for pain assessment are known in the art, including, for example, the Numerical Rating Scale, the Verbal Rating Scale, and the Brief Pain Inventory. Such tools could also be used to assess pain in accordance with the invention.

Additional indices for skin irritation may be used, including, e.g., the Draize Scale (hemorrhage, petechiae, erythema, edema, pruritus).

According to another aspect of the present invention, a preconfectioned injection device comprising a formulation according to the invention is provided. Such preconfectioned injection device adds additional benefits to the formulation according to the present invention, particularly with respect to ambulant administration, administration in areas of poor infrastructure, e.g., where non-medically skilled persons are responsible for the drug administration, or home use. Preferably, said preconfectioned injection device, such as a syringe, may hold a liquid volume of between ≧0.1 and ≦2 mL (single use), more preferably between 0.5 and 1.5 mL. Most preferred is an injection volume of about 0.8 or about 1.0 mL.

In accordance with the present invention it is to be understood that the pharmaceutical composition is also suitable for intramuscular administration or local administration. In a preferred embodiment of the present invention pharmaceutical composition is also suitable for intramuscular administration.

In a preferred embodiment, said preconfectioned injection device is either a (pre-filled) syringe, or an autoinjector. An autoinjector is a medical device designed to deliver one or more doses of a drug, particularly an injectable drug. Autoinjectors avoid the need of transferring a drug from a vial into an injection device—a step which laborious, often difficult and subject to particular risks (e.g., contamination or misdosage). Autoinjectors are easy to use and are intended for self-administration by patients, or administration by untrained personnel. Autoinjectors have a retractable needle, or a needle which is protected by a particular shield. Compared to syringes they offer facilitated handling, and they thus reduce risk of injury, or contamination, which contributes to their suitability for home use.

Autoinjectors further help to overcome the hesitation often associated with self-administration of the needle-based drug delivery device, and thus provide enhanced patient compliance, which in turn secures that the drug is regularly taken according to the prescribed dosage regimen, thus increasing the likelihood of therapeutic success. This is particularly important in therapeutic regimens which require repeated treatment, as is the case in many chronic diseases, like autoimmune diseases or in many cancer types which, due to targeted therapy, turn chronic nor near chronic.

Further, in such indications it is particularly beneficial if the patient can treat himself at home, as is the case with autoinjectors. Home treatment further reduces therapy costs and increases patient compliance, as the patients do not have to see medical personnel each time the dosage regimen requires that the drug is delivered. In a preferred embodiment of the invention, said autoinjector is from the spring-loaded syringe type. Such type contains a spring-loaded needle connected to a syringe. In another preferred embodiment, said autoinjector is from the gas jet autoinjector type. The latter contains a cylinder of pressurised gas and propels a fine jet of liquid through the skin without the use of a needle. This has the advantage that the autoinjector can be reloaded, and a variety of different doses or different drugs can be used. In another preferred embodiment of the invention, said preconfectioned injection device is selected from the group consisting of a conventional auto-injector, and/or wet/dry auto-injector.

A conventional autoinjector comprises the pharmaceutical formulation as outlined above and can be used for administration directly. A wet/dry auto-injector (also called “Liquid Dry autoinjector” or “Dual Chamber autoinjector”) is a two-chambered autoinjector that keeps the pharmaceutical formulation, or its active component, disposed in a dry chamber in a dry, stable form (e.g., lyophilized) until it is used. Prior to administration, the pharmaceutical formulation, or its active component, is reconstituted by transfer into a second chamber (“wet chamber”) containing a solvent or the solvent from a second chamber is transferred onto the first chamber. For said purpose, the dry chamber containing the solid medicament powder can for example also contain a volume of air or other gas which is replaced by the solvent when the pharmaceutical formulation, or its active component is reconstituted.

Preferably, the autoinjector is a disposable autoinjector and for single use. Suitable autoinjectors which can be used in the context of the present invention include the autoinjectors manufactured by Ypsomed. These include monodose devices, like the products sold under the trademarks “LyoTwist”, “YpsoMate”, “YpsoJect” and “VarioJect”.

Other suitable autoinjectors which can be used in the context of the present invention include the autocoinjectors manufactured by SHL. These include the products sold under the trademarks “Molly™”, “DAI™”, “DAI™-RNS”, “DAI™-R”, “SDI MIX+NIT™”, “VSDI™”, “PSDI™”, “Naisa™” and “DCP™ (OEM)”.

A further preferred type of autoinjector is the Physioject™ Disposable AutoInjector manufactured by Becton Dickinson. This auto-injector of the conventional type holds 1 ml prefilled syringes with a subcutaneous needle, is easy to assemble (2 assembly components), robust, has a large window for visual check and is tamper evident.

Another particularly preferred type of autoinjector is the BD™ Liquid Dry Injector manufactured by Becton Dickinson. This autoinjector of the wet/dry type allows the patient to reconstitute and inject a lyophilized pharmaceutical formulation according to the present invention, eliminating the need to handle vials and syringes.

Yet other suitable autoinjectors are the ASITMauto injector and the OTSTM disposable auto injector provided by BespakInjectables, the SafeClickTMautoinjector provided by Aqueo Future Injection technologies, and the SafeClickTM-Lyo and the SafeClickTM-Visco provided by Future Injection Technology. This list is however non-restricting.

In another preferred embodiment of the invention, the preconfectioned injection device is a pre-filled syringe or as syrette. A syrette is a device for injecting liquid through a needle. It is similar to a syringe except that it has a closed flexible tube instead of a rigid tube and piston. The term “pre-filled syringe” is self-explaining. Pre-filled syringes share many advantages with autoinjectors. Like autoinjectors, pre-filled syringes are available as conventional syringes and wet/dry syringes (also called dual-chamber syringes). Pre-filled syringes are, for example, provided by BoehringerIngelheim, Vetter Pharma International, Becton Dickinson, and others. According to a specific embodiment of the invention, an anti-TNFalpha antibody, such as adalimumab, or a biosimilar or interchangeable compound thereof, is applied via an autoinjector, such as a BD Physioject™ Disposable Autoinjector, using a formulation suitable for SC administration. An example for such formulation is the formulation according to the abovedescribed aspect of the invention, i.e. a formulation including acetate/acidic acid, arginine, and trehalose, preferably in the concentrations as set out before. Thus, the invention also includes such autoinjector device, comprising the anti-TNFalpha antibody prepared to be administered by such device.

According to another aspect of the present invention, a kit of parts is provided, said kit comprising at least a container comprising a pharmaceutical formulation as described before, and an injection device. The kit of parts or the preconfectioned injection device according to the invention is preferably adapted for subcutaneous administration. In such case, the injection needle has, preferably, a length of ≧10 mm to ≦100 mm and a gauge of between 0.2 mm and 1 mm (gauge 33 to 19).

According to yet another aspect of the present invention, the use of a formulation according to the invention, of a preconfectioned injection device according to the invention or of a kit of parts according to the invention for subcutaneous administration is provided.

According to yet another aspect of the present invention, the use of a formulation according to the invention, of a preconfectioned injection device according to the invention or of a kit of parts according to the invention for treatment of at least one disease selected from the group consisting of autoimmune disorders and/or malignant diseases is provided. Non-restricting examples for autoimmune disorders covered by said definition include rheumatoid arthritis, ankylosing spondylitis, inflammatory bowel disease, psoriasis, hidradenitissuppurativa and refractory asthma. Non-restricting examples for malignant diseases include NHL, breast cancer, CLL, metastatic colorectal cancer, non-squamous non-small cell lung cancer, glioblastoma, and metastatic renal cell carcinoma. In a further preferred embodiment the kit comprises instructions for subcutaneous or intramuscular administration of the formulation to a subject.

Naturally in view to the above, the present invention also relates to a method for reducing aggregation and/or fragmentation of a therapeutic monoclonal antibody using at least one of the compositions of the present invention, i.e. F3, F4 or F8 buffer. A skilled person will understand that formulating a therapeutically active antibody and/or fragment thereof which is susceptible to aggregation in one of the compositions and/or formulations and/or buffer of the present invention will lead to a reduced amount of aggregation compared to the formulation of a TNF-alpha antibody exhibiting the F1 formulation. Thus, in one aspect, the present invention relates to a method for reducing aggregation of a therapeutic monoclonal antibody, comprising formulating an antibody in a buffer selected from the group consisting of arginine-acetate buffer, pH 6.3 to 6.6, succinate buffer pH 6.1 to 6.4 and histidine buffer, pH 6.1 to 6.4, preferably further comprising trehalose or mannitol and evaluating any antibody aggregation before and after the antibody is formulated. An antibody which is “susceptible to aggregation” is one which has been found to aggregate with other antibody molecule(s), especially upon freezing and/or agitation. An antibody which is “susceptible to fragmentation” is one which has been found to be cleaved into two or more fragments, for example at a hinge region thereof.

By “reducing, aggregation, or fragmentation” is intended preventing or decreasing the amount of, aggregation, or fragmentation relative to the monoclonal antibody formulated at a different pH or in a different buffer.

These and other aspects of the invention will be apparent from and elucidated with reference to the embodiments described hereinafter.

Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

While the invention has been illustrated and described in detail in the drawings and foregoing description, such illustration and description are to be considered illustrative or exemplary and not restrictive; the invention is not limited to the disclosed embodiments.

Other variations to be disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting scope.

### EXAMPLES

Unless defined otherwise, all technical and scientific terms used herein have the same meaning as commonly understood by one of ordinary skill in the art (e.g., in cell culture, molecular genetics, nucleic acid chemistry, hybridization techniques, protein chemistry and biochemistry). Standard techniques are used for molecular, genetic and biochemical methods (see generally, Sambrook et a/., Molecular Cloning: A Laboratory Manual, 2^{nd} ed. (1989) Cold Spring Harbor Laboratory Press, Cold Spring Harbor, N.Y. and Ausubel et a/., Short Protocols in Molecular Biology (1999) 4^{th} Ed, John Wiley & Sons, Inc.—and the full version entitled Current Protocols in Molecular Biology, which are incorporated herein by reference) and chemical methods.

### Material:

NBT01 (=BI695501) product pool (bulk) #104601 at 8.2 mg/mL in 25 mM phosphate buffer, 212 mM NaCl, pH 5.7 was used. The experiments for the 24 weeks study analysis were performed at 2-8° C., 25° C. and 40° C. Result herein provided are the results of the different timepoints of t=0, 12, and 24 weeks.

### Analytical Assessment:

Appearance, pH, osmolarity, turbidity
Protein concentration (UV)
Particle size: PCS (photon correlation spectroscopy)
Protein integrity: HP-SEC (high performance size exclusion chromatography)
Binding activity: binding assay (Biacore), protein A and CD16 binding TNFα binding activity assay (Biacore; F1, F3, F8)

### Syringe Size and Filling:

0.84 mL in 1 mL glass syringe

### Example 1

### Evaluation of Formulation F8 Compared to Formulation F1 Exhibit Superior Features in Terms of Stability

Formulation studies have been carried out in which the formulation according to the invention (in the following: “F8”) was compared with a commercially available formulation of an anti TNFalpha antibody (IgG, humanized) (in the following: “F1”).

TABLE 2
Formulations subject to formulation studies

| No | Buffer | mmol/l | Tonifier 1 | mmol/l | Tonifier 2 | mmol/l | Surfactant | mmol/l | pH |
|---|---|---|---|---|---|---|---|---|---|
| F1 | Citrate + | 21.45 | Mannitol | 65.87 | NaCl | 105.45 | Polysorbate | 0.76 | 5.2 |
| | Phosphate | | | | | | 80 | | |
| F8 | Acetate + | 25 | Trehalose | 240 | none | n/a | Polysorbate | 0.76 | 6.5 |
| | Arginin | | | | | | 80 | | |

### Data Sampling

The formulations were stored under different storage conditions, and samples were drawn for analysis at different points of time. The following table shows the sampling of the stability study:

TABLE 3
Data sampling protocol

| Storage | | | 2 | 3 | 4 | 5 | 6 |
|---|---|---|---|---|---|---|---|
| Temp. | initial | 1 month | months | months | months | months | months |
| 2-8° C. | X | — | — | X | — | — | X |
| 25° C. | | — | — | X | — | — | X |
| 40° C. | | X | X | X | X | X | X |

### Concentration Changes Over Time

Concentration of the formulations was determined by diluting the drug product in duplicates 1 to 100 in the appropriate formulation buffer. The samples were measured in an UV/VIS spectrometer at 280 nm and at 320 nm (baseline correction) against the formulation buffer. From the measured values the concentration was calculated taking the mean of both samples. The results are shown in Table 4:

TABLE 4

| | | Concentration |
|---|---|---|
| Formulation | Sample point | [mg*mL⁻¹] |
| F1 | initial | 49.8 |
| | 4 weeks 40° C. | 49.1 |
| | 8 weeks 40° C. | 50.9 |
| | 12 weeks 40° C. | 49.6 |
| F8 | initial | 50.5 |
| | 4 weeks 40° C. | 50.7 |
| | 8 weeks 40° C. | 50.2 |
| | 12 weeks 40° C. | 50.6 |

As shown in Table 4, under the given storage conditions (0-12 weeks at 40° C.), formulation F8 exhibits smaller concentration changes over time than formulation F1.

### Osmolality Changes Over Time

Osmolality was determined with a freezing point osmometer. The osmolality of each formulation was calculated by measuring duplicates and taking the mean of the measured values. The results are shown in Table 5:

TABLE 5

| | | osmolality |
|---|---|---|
| Formulation | Sample point | [mOsmol*kg⁻¹] |
| F1 | initial | 324 |
| | 4 weeks 40° C. | 322 |
| | 8 weeks 40° C. | 320 |
| | 12 weeks 40° C. | 324 |
| F8 | initial | 308 |
| | 4 weeks 40° C. | 307 |
| | 8 weeks 40° C. | 310 |
| | 12 weeks 40° C. | 309 |

As shown in Table 5, under the given storage conditions (0-12 weeks at 40° C. storage), formulation F8 exhibits at least comparable osmolality stability over time as formulation F1.

### Turbidity Study

Turbidity of the formulation samples was determined with a turbidity photometer at 633 nm in duplicates. The mean of the two samples equaled the turbidity end value. The results are shown in Table 6:

TABLE 6

| | | Turbidity |
|---|---|---|
| Formulation | Sample point | (633 nm) [FNU] |
| F1 | initial | 24 |
| | 4 weeks 40° C. | 23 |
| | 8 weeks 40° C. | 23 |
| | 12 weeks 40° C. | 24 |
| F8 | initial | 8 |
| | 4 weeks 40° C. | 8 |
| | 8 weeks 40° C. | 8 |
| | 12 weeks 40° C. | 8 |

The results in Table 6 show that under the given storage conditions (0-12 weeks at 40° C. storage), formulation F8 exhibits less turbidity than formulation F1.

### Biacore Studies

Protein A binding activity, CD16V binding activity and TNFα binding activity was measured after different storage conditions. The results are shown in Table 7.

TABLE 7
Results of Biacore studies

| | | Binding of | Binding of | Binding of |
|---|---|---|---|---|
| Formulation | Sample point | protein A | CD16V | TNFα |
| F1 | initial | 100 | 100 | 111 |
| | 4 weeks 40° C. | 99 | 88 | |
| | 8 weeks 40° C. | 102 | 76 | |
| | 12 weeks 40° C. | 102 | 66 | 102 |
| | 12 weeks 25° C. | 106 | 100 | |
| | 12 weeks 2-8° C. | 103 | 98 | 124 |
| F8 | initial | 100 | 100 | 106 |
| | 4 weeks 40° C. | 98 | 95 | |
| | 8 weeks 40° C. | 96 | 89 | |
| | 12 weeks 40° C. | 99 | 84 | 105 |
| | 12 weeks 25° C. | 103 | 100 | |
| | 12 weeks 2-8° C. | 104 | 100 | 107 |

As can be seen from Table 7, formulation F8 does not change the binding properties and is therefore suitable as an alternative formulation providing for increased stability and shelf life, improved patient compliance by being adapted to SC administration (with respect to parameters such as concentration, osmolarity, and viscosity) and by avoiding the use of citric acid as a buffer component, which is assumed to provide for significant needle pain.

### Example 2

### Evaluation of Formulation of F3, F4 and F8 Compared to F1 in Terms of Stability

Formulation studies have been conducted in which the formulations according to the invention (in the following: “F3”, “F4”, “F8”) were compared with a commercially available formulation of an anti TNFalpha antibody (humanized IgG) (in the following: “F1”). The antibody was transferred into the listed buffers by dialysis using Slide-A-Lyzer cassettes with a molecular weight cutoff at 10 kDa (Thermo Scientific). Polysorbate was spiked into the solution after dialysis.

TABLE 8
Formulation compositions

| No | Buffer | mmol/l | Tonifier 1 | mmol/l | Tonifier 2 | mmol/l | Surfactant | mmol/l | pH |
|---|---|---|---|---|---|---|---|---|---|
| F1 | Citrate + | 21.45 | Mannitol | 65.87 | NaCl | 105.45 | Polysorbate | 0.76 | 5.2 |
| | Phosphate | | | | | | 80 | | |
| F3 | Succinate | 25 | Trehalose | 215 | none | n/a | Polysorbate | 0.76 | 6.25 |
| | | | | | | | 80 | | |
| F4 | Histidine | 25 | Mannitol | 240 | none | n/a | Polysorbate | 0.76 | 6.25 |
| | | | | | | | 80 | | |
| F8 | Acetate + | 25 | Trehalose | 240 | none | n/a | Polysorbate | 0.76 | 6.5 |
| | Arginine | | | | | | 80 | | |

### Formulation Sampling

For a stability study, formulations were stored in syringes at different temperatures and samples were withdrawn for analysis at different time points according to Table 9.

TABLE 9
Sampling protocol for stability study

| Storage | | | | 12 | 16 | 20 | 24 |
|---|---|---|---|---|---|---|---|
| Temp. | initial | 4 weeks | 8 weeks | weeks | weeks | weeks | weeks |
| 2-8° C. | X | — | — | X | — | — | X |
| 25° C. | | — | — | X | — | — | X |
| 40° C. | | X | X | X | X | X | X |

### Monitoring Protein Concentration Over Time

Protein concentrations were determined by diluting the drug product in duplicates by a factor of 100 in the appropriate formulation buffer. Protein concentrations were measured using a UV/VIS spectrometer (Lambda 35, Co. Perkin Elmer) at a wavelength of 280 nm. Protein concentrations were calculated as the mean of the duplicates. Table 10 exhibits protein concentrations in formulations F1, F3, F4, and F8 withdrawn from samples stored at a temperature of 40° C.

TABLE 10
Protein concentration time course at a temperature of 40° C.

| | | Concentration |
|---|---|---|
| Formulation | Sampling point | [mg/mL] |
| F1 | initial | 49.8 |
| | 4 weeks 40° C. | 49.1 |
| | 8 weeks 40° C. | 50.9 |
| | 12 weeks 40° C. | 49.6 |
| | 16 weeks 40° C. | 51.2 |
| | 20 weeks 40° C. | 52.0 |
| | 24 weeks 40° C. | 52.5 |
| F3 | initial | 50.5 |
| | 4 weeks 40° C. | 48.7 |
| | 8 weeks 40° C. | 51.1 |
| | 12 weeks 40° C. | 50.1 |
| | 16 weeks 40° C. | 51.5 |
| | 20 weeks 40° C. | 51.4 |
| | 24 weeks 40° C. | 52.3 |
| F4 | initial | 51.4 |
| | 4 weeks 40° C. | 49.9 |
| | 8 weeks 40° C. | 52.9 |
| | 12 weeks 40° C. | 50.7 |
| | 16 weeks 40° C. | 52.2 |
| | 20 weeks 40° C. | 52.9 |
| | 24 weeks 40° C. | 53.2 |
| F8 | initial | 50.5 |
| | 4 weeks 40° C. | 50.7 |
| | 8 weeks 40° C. | 50.2 |
| | 12 weeks 40° C. | 50.6 |
| | 16 weeks 40° C. | 52.4 |
| | 20 weeks 40° C. | 52.1 |
| | 24 weeks 40° C. | 54.0 |

Under the given experimental conditions (storage for 0-24 weeks at a temperature of 40° C.) formulations F1, F3, F4, and F8 exhibit similar protein concentrations at the respective time points.

### Monitoring Osmolality Over Time

Osmolalities of formulations F1, F3, F4, and F8 were determined in duplicates using a freezing point osmometer (Osmomat 030, Co. Gonotec). The osmolality of each formulation was calculated as the mean of the duplicates by measuring the undiluted formulations at a sample volume of 504 each. The freezing point temperatures were determined by cooling the sample to a final temperature of −7° C. Table 11 exhibits osmolalities of formulations F1, F3, F4, and F8 withdrawn from samples stored at a temperature of 40° C.

TABLE 11
Protein concentration time course at a temperature of 40° C.

| | | Osmolality |
|---|---|---|
| Formulation | Sampling point | [mOsmol/kg] |
| F1 | initial | 324 |
| | 4 weeks 40° C. | 322 |
| | 8 weeks 40° C. | 320 |
| | 12 weeks 40° C. | 324 |
| | 16 weeks 40° C. | 328 |
| | 20 weeks 40° C. | 328 |
| | 24 weeks 40° C. | 325 |
| F3 | initial | 318 |
| | 4 weeks 40° C. | 320 |
| | 8 weeks 40° C. | 318 |
| | 12 weeks 40° C. | 324 |
| | 16 weeks 40° C. | 320 |
| | 20 weeks 40° C. | 323 |
| | 24 weeks 40° C. | 321 |
| F4 | initial | 306 |
| | 4 weeks 40° C. | 307 |
| | 8 weeks 40° C. | 307 |
| | 12 weeks 40° C. | 306 |
| | 16 weeks 40° C. | 312 |
| | 20 weeks 40° C. | 309 |
| | 24 weeks 40° C. | 307 |
| F8 | initial | 308 |
| | 4 weeks 40° C. | 307 |
| | 8 weeks 40° C. | 310 |
| | 12 weeks 40° C. | 309 |
| | 16 weeks 40° C. | 316 |
| | 20 weeks 40° C. | 313 |
| | 24 weeks 40° C. | 311 |

Under the given experimental conditions (storage for 0-24 weeks at a temperature of 40° C.) the osmolality of formulations F1, F3, F4, and F8 is equally stable.

Monitoring turbidity over time Turbidities of the formulation F1, F3, F4, and F8 were determined from undiluted samples at a volume of 120 μL at a protein concentration 150 mg/ml in duplicates using a turbidity photometer (Co. Boehringer-IngelheimPharma GmbH & Co. KG in collaboration with Co. Microparts) by light scattering at a wavelength of 633 nm. The turbidity of each formulation was calculated as the mean of the duplicates. Table 12 exhibits the turbidities of formulations F1, F3, F4, and F8 withdrawn from samples stored at a temperature of 40° C.

TABLE 12
Turbidity time course at a temperature of 40° C.

| | | Turbidity |
|---|---|---|
| Formulation | Sampling point | (633 nm) [FNU] |
| F1 | initial | 24 |
| | 4 weeks 40° C. | 23 |
| | 8 weeks 40° C. | 23 |
| | 12 weeks 40° C. | 24 |
| | 16 weeks 40° C. | 25 |
| | 20 weeks 40° C. | 26 |
| | 24 weeks 40° C. | 26 |
| F3 | initial | 21 |
| | 4 weeks 40° C. | 20 |
| | 8 weeks 40° C. | 20 |
| | 12 weeks 40° C. | 20 |
| | 16 weeks 40° C. | 21 |
| | 20 weeks 40° C. | 21 |
| | 24 weeks 40° C. | 21 |
| F4 | initial | 7 |
| | 4 weeks 40° C. | 7 |
| | 8 weeks 40° C. | 8 |
| | 12 weeks 40° C. | 8 |
| | 16 weeks 40° C. | 9 |
| | 20 weeks 40° C. | 9 |
| | 24 weeks 40° C. | 10 |
| F8 | initial | 8 |
| | 4 weeks 40° C. | 8 |
| | 8 weeks 40° C. | 8 |
| | 12 weeks 40° C. | 8 |
| | 16 weeks 40° C. | 9 |
| | 20 weeks 40° C. | 9 |
| | 24 weeks 40° C. | 9 |

Under the given experimental conditions (storage for 0-24 weeks at a temperature of 40° C.) turbidity values for formulations F3 and in particular F4 and F8 are lower as compared to the originator formulation F1.

### Monitoring Protein Activity Over Time

Protein function was monitored at different storage conditions by diluting the samples to a concentration of 0.6 mg/mL at protein concentration 150 mg/ml in assay buffer (0.01 M HEPES, 0.15 M NaCl, 3 mM EDTA and 0.005% PS-20). Three different types of binding were monitored, Protein A binding was measured to determine if the protein was correctly folded and functional, then both CD16 and TNFα binding were measured for functional activity. This testing was carried out using a Biacore instrument (GE Healthcare). The percent response in comparison to the standard sample is reported in Table 13. Table 13 exhibits binding activities of formulations F1, F3, F4, and F8 withdrawn from samples stored at a temperature of 40° C., 25° C., or 2-8° C.

TABLE 13
Protein activity time course

| | | Binding of | Binding of | Binding of |
|---|---|---|---|---|
| | | protein A | CD16V | TNFα |
| Formulation | Sampling point | [%] | [%] | [%] |
| F1 | initial | 100 | 100 | 111 |
| | 4 weeks 40° C. | 99 | 88 | |
| | 8 weeks 40° C. | 102 | 76 | |
| | 12 weeks 40° C. | 102 | 66 | 102 |
| | 12 weeks 25° C. | 106 | 100 | |
| | 12 weeks 2-8° C. | 103 | 98 | 124 |
| | 16 weeks 40° C. | 110 | 57 | |
| | 20 weeks 40° C. | 97 | 49 | |
| | 24 weeks 40° C. | 95 | 43 | 94 |
| | 24 weeks 25° C. | 99 | 94 | |
| | 24 weeks 2-8° C. | 102 | 100 | 107 |
| F3 | initial | 100 | 100 | 107 |
| | 4 weeks 40° C. | 96 | 92 | |
| | 8 weeks 40° C. | 101 | 81 | |
| | 12 weeks 40° C. | 100 | 77 | 115 |
| | 12 weeks 25° C. | 101 | 100 | |
| | 12 weeks 2-8° C. | 99 | 98 | 116 |
| | 16 weeks 40° C. | 110 | 68 | |
| | 20 weeks 40° C. | 102 | 61 | |
| | 24 weeks 40° C. | 86 | 56 | 100 |
| | 24 weeks 25° C. | 109 | 96 | |
| | 24 weeks 2-8° C. | 106 | 99 | 107 |
| F4 | initial | 100 | 100 | 120 |
| | 4 weeks 40° C. | 104 | 93 | |
| | 8 weeks 40° C. | 105 | 84 | |
| | 12 weeks 40° C. | 99 | 77 | 102 |
| | 12 weeks 25° C. | 101 | 101 | |
| | 12 weeks 2-8° C. | 100 | 100 | 111 |
| | 16 weeks 40° C. | 99 | 73 | |
| | 20 weeks 40° C. | 95 | 67 | |
| | 24 weeks 40° C. | 98 | 62 | |
| | 24 weeks 25° C. | 107 | 99 | |
| | 24 weeks 2-8° C. | 104 | 96 | |
| F8 | initial | 100 | 100 | 106 |
| | 4 weeks 40° C. | 98 | 95 | |
| | 8 weeks 40° C. | 96 | 89 | |
| | 12 weeks 40° C. | 99 | 84 | 105 |
| | 12 weeks 25° C. | 103 | 100 | |
| | 12 weeks 2-8° C. | 104 | 100 | 107 |
| | 16 weeks 40° C. | 101 | 78 | |
| | 20 weeks 40° C. | 102 | 71 | |
| | 24 weeks 40° C. | 99 | 67 | 88 |
| | 24 weeks 25° C. | 101 | 98 | |
| | 24 weeks 2-8° C. | 101 | 100 | 107 |

At a temperature of 2-8° C., binding properties of formulation F1, F3, F4, and F8 do not change over the course of 24 weeks. At temperatures of 25° C. or 40° C., binding properties of formulation F3, F4, and F8 are either similar or higher (bold numbers, Table 13) as compared to F1 over the course of 24 weeks, signifying similar or better function than the F1 formulation.

Monitoring particle size over time Mean particle sizes (Z-average values) in the formulations F1, F3, F4, and F8 were determined by dynamic light scattering using a ZetasizerNanoZS ZEN3600 (Malvern Instruments). Two undiluted samples AT a volume of 75 μL in a single-use cuvette were irradiated with a helium-neon laser at a wavelength of 633 nm and a temperature of 20° C. (Table 14).

TABLE 14
Particle size time course at a temperature of 40° C.

| | | Hydrodynamic |
|---|---|---|
| Formulation | Sampling point | diameter [nm] |
| F1 | initial | 16 |
| | 12 weeks 40° C. | 15 |
| | 24 weeks 40° C. | 17 |
| F3 | initial | 19 |
| | 12 weeks 40° C. | 18 |
| | 24 weeks 40° C. | 19 |
| F4 | initial | 10 |
| | 12 weeks 40° C. | 10 |
| | 24 weeks 40° C. | 11 |
| F8 | initial | 12 |
| | 12 weeks 40° C. | 11 |
| | 24 weeks 40° C. | 12 |

Mean particle sizes of all formulations do not change over the course of 24 weeks when stored at a temperature of 40° C. Mean particle sizes of formulation F1 and F3 are greater as compared to those in formulations F4 and F8.

In summary, all described formulations can be considered as being suitable as alternatives to the originator formulation with respect to providing increased stability, shelf life, improved patient compliance if adapted to formulations for s.c. administration. An additional benefit of the alternative formulations as compared to the originator formulation F1 can be the avoidance of citric acid as a buffer component, which may avoid local pain at the injection site.

### Example 3

### Pain Perception after Subcutaneous Injections of Formulations F1 Compared F3, F4 and F8

In the study of Laursen et al 2006, Basic & Clinical Pharmacology & Toxicology, 98, 218-221 it had been significantly found that the citrate buffer in which erythropoietin as a pharmaceutical active substance caused more pain than the histidine buffer immediately after injection (PΩ0.002) in more participants (38/54). Histidine buffer did not cause more pain than saline (PSΩ0.996). Thus, it is prudent to expect that in an analogous fashion also a histidine-buffered TNF alpha antibody will cause less pain upon injection. For evaluating the perception of pain by subcutaneous injection of histidine, arginine-acetate and/or succinate buffered compositions such as F3, F4 and F8 of Example 1 and 2 and commercially available solutions such as F1 for dispensing TNF alpha healthy volunteers (mean age (∫S.E.M.): 35.5∫1.1 years) are recruited to a double-blind, randomized study.

### Experimental Design.

The study can be carried out in a double-blind, randomized design. The perception of pain can be evaluated after subcutaneous injection in the thigh of three different test compositions (as shown in table 8). The injection volume can be 0.3 ml on all occasions, 30 G, 8 mm needles can be employed, and all injections can be performed in a 45 an angle in a lifted skin-fold. Injections can be given pair-wise, first one in the right thigh, and immediately after one in the left thigh, in all two pairs of injections (4 injections). Preferably a 5 min. interval will be given between the two pairs of injections. Injection pairs can be given in random order. Randomizing and blinding can be performed by the Dispensary of the hospital. All participants can receive at least one injection pair including A and D, and one injection pair including A and E and optionally A and B and/or A and C. The same applies for buffer B and C, i.e. B and D, B and E; C and D and C and E optionally A and E. All injections in each subject can be performed in one day, and by the same experienced nurse, assuring that e.g. the rate of injection is kept constant. After each pair of injection the participants evaluated, using a 5 point verbal rating scale (VRS) (see legends for figures), whether they experienced much more or more pain after one of the injections within the pair, or if there is no difference between the two injections within the pair. The VRS has been used in many studies of pain perception (Frenken et al. 1993; Jorgensen 1994; Grond et al. 1995). Evaluation of pain can be made immediately after injection of each pair and 2 min. after injections.

In this context, A could be the formulation F3, B could be the formulation F4, C could be the formulation F8,D could be the formulation F1, and optionally E could be a saline formulation.

### Statistics.

A one-sided binomial test with a 5% significance level can be applied to assess if one buffer caused more pain than the other. With no difference between buffers, the distribution can be assumed to be symmetric around “score 3”. The statistical results are reported according to the null hypotheses: i) solution B does not cause more pain than A, and ii) solution A does not cause more pain than C. As the most conservative test, the one-sided test can be performed against the alternative that the probability of more or much more pain is _{—}50%.

1: An essentially citrate- and/or phosphate-free aqueous buffer solution designed for administration of a therapeutically active antibody or antibody fragment thereof, wherein the antibody is used for the treatment of autoimmune or malignant diseases, the buffer comprising: greater than 5 mg/ml antibody, and having a pH value of at least 5.5.

2: The buffer of claim 1, wherein the solution comprising at least:
i) histidine, and
mannitol
in pharmaceutically acceptable quantities; or
ii) succinate, and
trehalose,
in pharmaceutically acceptable quantities; or
iii) acetate, and/or acidic acid
arginine, and
trehalose
in pharmaceutically acceptable quantities and at a pharmaceutically acceptable pH.

3: A pharmaceutical composition comprising
i) a therapeutic antibody or antibody fragment thereof in a concentration of from 5 to 200 mg/ml,
histidine in a concentration of from 10 to 200 mmol l⁻¹,
mannitol in a concentration of from 50 to 1000 mmol l⁻¹,
and optionally a surfactant, at a pH value in the range of 5.5 to 8, of 6 to 7, or
ii) a therapeutic antibody or antibody fragment thereof in a concentration of from 5 to 200 mg/ml,
succinate in a concentration of from 10 to 200 mmol l⁻¹,
trehalose in a concentration of from 50 to 1000 mmol l⁻¹,
and optionally a surfactant, at a pH value in the range of 5.5 to 8, of 6 to 7; or
iii) a therapeutic antibody or antibody fragment thereof in a concentration of from 5 to 200 mg/ml,
acetate and/or acidic acid in a concentration of from 10 to 200 mMol l⁻¹,
arginine in a concentration of from 5 to 100 mMol l⁻¹;
trehalose in a concentration of from 50 to 1000 mMol l⁻¹,
and optionally a surfactant, at a pH value in the range of 5.5 to 8.

4: The pharmaceutical composition according to claim 2, wherein the therapeutic antibody is an anti-TNF alpha antibody, and the composition comprising:
i) the anti-TNF alpha antibody in a concentration of 50 mg/ml,
histidine in a concentration of 25 mmol l⁻¹, and
mannitol in a concentration of 240 mmol l⁻¹; or
ii) the anti-TNF alpha antibody in a concentration of 50 mg/ml,
succinate in a concentration of 25 mmol l⁻¹, and
trehalose in a concentration of 215 mmol l⁻¹; or
iii) the anti-TNF alpha antibody in a concentration of 50 mg/ml,
acetate and/or acidic acid in a concentration of 15 mMol l⁻¹,
arginine in a concentration of 10 mMol l⁻¹, and
trehalose in a concentration of 240 mMol l⁻¹;
wherein the compositions of i) and ii) having a pH value of 6.25+/−0.5 and the composition in iii) having a pH value of 6.5+/−0.5.

5: The pharmaceutical composition according to claim 3, further comprising a surfactant, optionally selected from the group consisting of polysorbate 20 and polysorbate 80, and optionally in an amount of 0.001%-10.0% m/v.

6: The pharmaceutical composition according to claim 3, having a pH value of from 5.5 to 8.

7: The pharmaceutical composition according to claim 5, wherein said therapeutic antibody or said anti-TNFalpha antibody is selected from the group consisting of adalimumab, infliximab, certolizumabpegol, golimumab, and antibodies being biosimilar or interchangeable with respect to adalimumab, infliximab, certolizumabpegol, or golimumab.

8: The pharmaceutical composition according to claim 6, wherein the composition is adapted for subcutaneous administration.

9: The pharmaceutical composition according to claim 7, wherein the composition is stable at 40° C.+/−3° C. for at least 3 months.

10: The pharmaceutical composition according to claim 8, wherein the composition has at least one feature selected from the group consisting of:
(i) Increased shelf life
(ii) better temperature stability, and/or
(iii) decreased formation of aggregates
compared to a formulation comprising citrate and phosphate as buffers, and mannitol as tonifier.

11: The pharmaceutical composition according to claim 9, wherein the composition having at least one feature selected from the group consisting of:
(a) decreased amount of aggregates as measured by High Pressure Size Exclusion Chromatography (HP-SEC),
(b) higher amount of monomers after storage at about 40° C. as measured by HP-SEC
(c) significant less fragments as measured by HP-SEC,
(d) smaller hydrodynamic diameter in Z-Average (nm) at 24 weeks and/or less increase in Polydispersity Index (PDI) at storage at 40° C.
(e) increased binding activity as measured by Biacore after storage at about 40° C., and/or
(f) lower turbidity value in FormazinNephelometry Units (FNU), compared to a reference composition.

12: The pharmaceutical composition according to claim 8, wherein composition i) and/or iii) exhibiting a Z-average (nm) of below 12+/−1 and a PDI of below 0.6+/−0.2 and/or wherein said formulation is substantially free from particulates upon storage at about 5° C. for at least 6 months as determined by visual inspection.

13: The pharmaceutical composition according to claim 8, wherein composition i) and/or ii) exhibiting a turbidity value in FormazinNephelometry Units (FNU) below 10 and/or composition ii) exhibiting a turbidity below 20 FNU.

14: The pharmaceutical composition according to claim 8, wherein said antibody or fragment thereof retains at least 90% of binding ability to a transforming growth factor alpha and/or CD16 polypeptide compared to a reference antibody preparation.

15: The pharmaceutical composition according to claim 8, wherein less than 5%+/−0.5% of said antibody or fragment thereof forms an aggregate upon storage at about 40° C. for at least 6 months as determined by HP SEC.

16: The pharmaceutical composition according to claim 8, wherein less than 3%+/−0.5% of said antibody or fragment thereof is fragmented upon storage at about 40° C. for at least 6 months as determined by HP SEC.

17: The pharmaceutical composition according to claim 8, wherein less than 91.7%+/−0.5% of said antibody or fragment thereof is monomeric upon storage at about 40° C. for at least 6 months as determined by HP SEC.

18: The pharmaceutical composition according to claim 16, designed for the subcutaneous administration and/or for use in the treatment of a disease selected from the group consisting of autoimmune disorders and malignant diseases.

19: The pharmaceutical composition according to claim 17, wherein injection of the composition reduces pain associated with the injection designed to be administrated in a subject.

20: A preconfectioned injection device comprising an aqueous buffer solution according to claim 1.

21: The preconfectioned injection device according to claim 20, wherein said device is an autoinjector or a pre-filled syringe.

22: The preconfectioned injection device according to claim 20, wherein said composition is suitable for subcutaneous administration or intramuscular administration.

23: A kit of parts, comprising at least a container comprising a pharmaceutical composition according to claim 2, and an injection device.

24: The kit of claim 23, further comprising instructions for subcutaneous or intramuscular administration of the formulation to a subject.

25: A method for reducing aggregation and/or fragmentation of a therapeutic monoclonal antibody, comprising formulating an antibody in a buffer selected from the group consisting of arginine-acetate buffer, pH 6.3 to 6.6, succinate buffer pH 6.1 to 6.4 and histidine buffer, pH 6.1 to 6.4, and evaluating any antibody aggregation before and after the antibody is formulated.

26: The method according to claim 25 wherein the buffer further comprises trehalose or mannitol.
